(19) United States
(12) Patent Application Publication
(10) Pub. No.: US 2008/0138822 A1
(43) Pub. Date: Jun. 12, 2008

(54) Differentiating nucleic acids with ultraviolet radiation

(76) Inventor: Roy L. Rike, (US)

Correspondence Address: ROY L. RIKE, 9 OAK ST, APT C, BRUNSWICK, ME, 04011, US

(21) Appl. No.: 11/983,666

(22) Filed: Nov. 10, 2007

Related U.S. Application Data
(60) Provisional application No. 60/873,838, filed on Dec. 8, 2006.

(51) Int. Cl. C12Q 1/68; G06F 19/00

(52) U.S. Cl. 435/6; 702/20

(57) ABSTRACT

A method of using ultraviolet radiation to differentiate nucleic acids (DNA, RNA) with particular application to forensic science. The illustrative embodiment shows an ultraviolet radiation reactor made of a block (10) fitted with an optical chamber (12) and sample tube insertion hole (14). A UV light source (16) is fitted to the chamber. The sample tube containing the nucleic acid is placed in the insertion hole and the light source activated for a predetermined time. The exposed sample is then removed and undergoes a further laboratory assay, typically spectrophotometric, to reveal the distinctive “signature” induced upon the acid by the ultraviolet irradiation.

### CROSS REFERENCE TO RELATED APPLICATIONS

This application claims priority in provisional application filed on Dec. 8, 2006, entitled “Differentiating Nucleic Acids With Ultraviolet Radiation,” serial No. 60/873,838, by inventor Roy L. Rike.

### FEDERALLY SPONSORED RESEARCH

Not Applicable

### SEQUENCE LISTING OR PROGRAM

Not Applicable

### BACKGROUND OF THE INVENTION

This invention relates to differentiating nucleic acids (DNA, RNA) specifically as such differentiation uses ultraviolet radiation to create peculiar and characterizable molecular formations upon the acids.

### BACKGROUND OF THE INVENTION

There is currently great interest in developing new methods of genetic profiling which are faster, less costly, and less labor intensive. Pressure to process and analyze growing numbers of samples of genetic material is placing forensic laboratories under severe stress. The introduction of a simple “front end” method of analysis will help forensic scientists save time by allowing them to tell quickly whether genetic samples are of probative, or non-probative, value. The 2007 Solicitation of the U.S. Department of Justice for forensic DNA research and development proposals (CFDA No. 16.560) attests to the current need for such new forensic methods of DNA analysis.

At the present time there is no simple “front end” method of forensic DNA analysis which would allow a preliminary triage of genetic samples. The method described in my patent fills this gap by exploiting a well-studied microbiological phenomenon, the formation of certain molecular formations, or photoproducts, such as pyrimidine and pyrimidone dimers, in specific areas of nucleic acids which are exposed to ultraviolet (UV) radiation (see Mutation Research, C. Auerbach, Chapman and Hall, London, 1976; The Biology of Cancer, R. A. Weinberg, Garland Science, New York, 2007). Insofar as such molecular formations form distinctively according to the unique sequence of bases in each nucleic acid, ultraviolet radiation is capable of generating genetic “signatures” which will allow forensic scientists to distinguish genetic samples by classifying the distribution and concentration of these photoproducts. The simple process of inducing damage to the nucleic acid with a dose of ultraviolet radiation becomes a method of gene profiling.

Yet forensic scientists have not used UV photoproducts from the field of mutation research for application to their own DNA “fingerprinting”. Mutation research scientists for their part have restricted their interested in ultraviolet radiation to the study of cellular damage and repair mechanisms: UV is damage and distortion, not information. Accordingly, a search of prior art indicates that no one has intentionally employed ultraviolet radiation to induce the creation of distinctive molecular formations upon nucleic acids for the purpose of deriving forensic “fingerprints”. Within the patent field of genetic profiling, ultraviolet radiation is currently claimed only in a decontaminating role or as an adhesive to fix DNA fragments to a filter. See U.S. Pat. No. 5,474,911 to Pontius (1995), U.S. Pat. No. 5,747,254 to Pontius (1995), and U.S. Pat. No. 6,238,866 to Yeh and Wilkins (2001).

### OBJECTS AND ADVANTAGES

The method described in my patent creates a new type of genetic fingerprint by irradiating samples of nucleic acids with an identical dose of ultraviolet radiation and then subjecting the samples to additional microbiological assays such as spectrophotometric analysis (see Absorption Spectroscopy of Organic Molecules, V. M. Parikh, Addison-Wesley Publishing Company, Reading, Mass., 1974). The resulting graphs and numerical information recording the UV-induced molecular formations can then be compared and classified in the manner of fingerprints. The method thereby becomes the simple “front end” analysis missing from forensic genetic analysis and which it now requires. Scientists using the method can quickly rule out non-probative samples and devote their time to those that promise better evidentiary value.

### SUMMARY

In accordance with the present invention, a method of employing ultraviolet radiation to differentiate nucleic acids comprising exposing the acids to wavelengths of radiation sufficient to induce the creation of distinguishing molecular formations. The distribution and concentration of these formations as subsequently revealed by an instrument such as a spectrophotometer permits samples of the acids to be identified and classified.

### DRAWINGS

### Figures

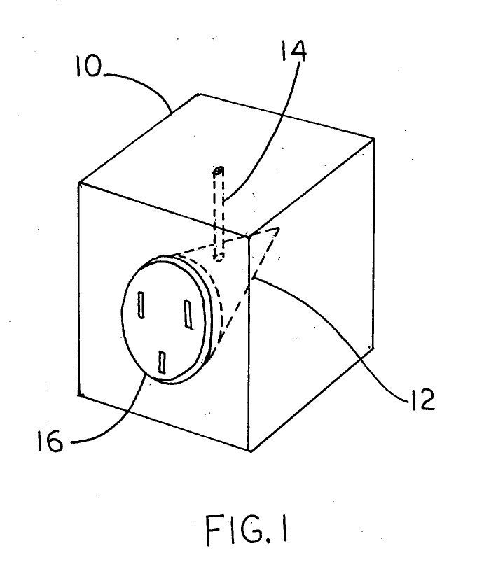

FIG. 1 shows an illustrative ultraviolet radiation reactor constructed by the method.

### REFERENCE NUMERALS

10 block
12 optical chamber
14 sample tube insertion hole
16 light source

### DETAILED DESCRIPTION

### FIG. 1—Illustrative Embodiment

An illustrative embodiment of the method described in the application is illustrated in FIG. 1 (overall view). A block of material is formed 10 containing an optical chamber 12 (here, conical) and a sample tube insertion hole 14. The optical chamber 12 also holds the (UV light source 16 (here, a light emitting diode). The material of the block 10 will typically be of ceramic material and constructed with dimensions to suit those of the light source 16 and the dimensions of the sample tube to be placed in the insertion hole 14. The external dimensions of the illustrative free-standing device will be sufficient to ensure the physical stability of the device.

### Operation—FIG. 1

A sample tube containing nucleic acid is inserted into sample tube insertion hole 14. The UV light source 16 is turned on for a predetermined length of time. When the irradiation complete, the sample is removed and subjected to additional biological assays, typically spectrophotometric analysis, which will graphically and numerically record the presence of distinctive and classifiable molecular formations induced by the irradiation.

### Advantages

From the description above, the advantages of my method of differentiating nucleic acids are evident in the method's simplicity, rapidity, and economy. Molecular biologists, particularly those working in the forensic sciences, can employ ultraviolet radiation reactors constructed by this method to perform rapid preliminary evaluations of samples of genetic material.

### CONCLUSION, RAMIFICATIONS, AND SCOPE

Accordingly, the reader will see that the method of differentiating nucleic acids with ultraviolet radiation described in this invention will provide the simple “front end” analysis missing from genetic forensic analysis. These scientists will now be better able to cope with the increasing workloads created by the demand for genetic analysis. More broadly, the method will make the laboratory work of all molecular biologists more efficient for the same reasons of simplicity, rapidity, and economy.

The illustrative embodiment described in this application is an ultraviolet radiation reactor used to show the principles of the method which I have invented and should not be construed as limiting the scope of the method. The scope of the method should be determined by the appended claim and its legal equivalents rather than by the example given.

I claim

1. A method of employing ultraviolet radiation to differentiate nucleic acids, Comprising:
(a) exposing said acids to wavelengths of ultraviolet radiation for durations of time sufficient to create distinguishing molecular formations upon them, the distribution and concentration of which may be used to identify and characterize the acids,
whereby the identification of genetic materials may be assisted with simple machinery and made more rapid and efficient.
